(19) United States
(12) Patent Application Publication
(10) Pub. No.: US 2012/0109207 A1
(43) Pub. Date: May 3, 2012

(54) Enhanced Interfacial Conformance for a Composite Rod for Spinal Implant Systems with Higher Modulus Core and Lower Modulus Polymeric Sleeve

(75) Inventor: Hai H. Trieu, Cordova, TN (US)

(73) Assignee: WARSAW ORTHOPEDIC, INC., Warsaw, IN (US)

(21) Appl. No.: 12/915,892

(22) Filed: Oct. 29, 2010

(51) Int. Cl. A61B 17/70; B23P 11/00

(52) U.S. Cl. 606/254; 29/428

(57) ABSTRACT

A spinal rod includes a core component and a tube. The core component has a diameter and an axial length. The tube has a diameter equal to or less than the diameter of the core component. A vibrational energy is applied between the core and the tube such that the core is received within the tube and the tube is advanced along the axial length of the tube. The spinal rod composite then has facial conformance forces maintaining the tube position along the axial length of the core.

### FIELD OF INVENTION

Embodiments of the invention relate to spinal fixation systems having at least one composite component. More particularly, the embodiments relate to rods for use in spinal fixation systems that are composites of polyetheretherketone (PEEK) and metals or metal alloys.

### BACKGROUND

The spinal column is a biomechanical structure composed primarily of support structures including vertebrae and intervertebral discs and soft tissue structures for motive and stabilizing forces including muscles and ligaments. The biomechanical functions of the spinal column include support, spinal cord protection, and motion control between the head, trunk, arms, pelvis, and legs. These biomechanical functions may require oppositely designed structures. For example, the support function may be best addressed with rigid load bearing structures while motion control may be best suited for structures that are easily movable relative to each other. The trade-offs between these biomechanical functions may be seen within the structures that make up the spinal column. Damage to one or more components of the spinal column, such as an intervertebral disc, may result from disease or trauma and cause instability of the spinal column and damage multiple biomechanical functions of the spinal column. To prevent further damage and overcome some of the symptoms resulting from a damaged spinal column, a spinal fixation device may be installed to stabilize the spinal column.

A spinal fixation device generally consists of stabilizing elements, such as rods or plates, attached by anchors to the vertebrae in the section of the vertebral column that is to be stabilized. The spinal fixation device restricts the movement of the vertebrae relative to one another and supports at least a part of the stresses created by the weight of the body otherwise imparted to the vertebral column. Typically, the stabilizing element is rigid and inflexible and is used in conjunction with an intervertebral fusion device to promote fusion between adjacent vertebral bodies. There are some disadvantages associated with the use of rigid spinal fixation devices, including decreased mobility, stress shielding (i.e. too little stress on some bones, leading to a decrease in bone density), and stress localization (i.e. too much stress on some bones, leading to fracture and other damage).

In response, flexible spinal fixation devices have been employed. These devices are designed to support at least a portion of the stresses imparted to the vertebral column but also allow a degree of movement. In this way, flexible spinal fixation devices avoid some of the disadvantages of rigid spinal fixation devices. These devices may be made of a material having a lower modulus of elasticity, or by combining materials in complex manufacturing processes to create composites having more flexibility.

The description herein of problems and disadvantages of known apparatuses, methods, and devices is not intended to limit the invention to the exclusion of these known entities. Indeed, embodiments of the invention may include, as a part of the embodiment, portions or all of one or more of the known apparatus, methods, and devices without suffering from the disadvantages and problems noted herein.

### SUMMARY

An embodiment of the invention includes a spinal rod having a core component and a tube. The core component has a diameter and an axial length. The tube has a diameter equal to or less than the diameter of the core component. A vibrational energy is applied between the core and the tube such that the core is received within the tube and the tube is advanced along the axial length of the tube. The spinal rod composite then has facial conformance forces maintaining the tube position along the axial length of the core.

Another embodiment of the invention may include a method of forming a composite rod. A step may include advancing a core into a tube. The tube has an inner diameter less than the diameter of the core. Another step may include vibrating the core relative to the tube as the advancing step occurs.

Yet another embodiment of a spinal rod may include a core component, a first tube, and a second tube. The core component has a first diameter extending along a first length of the core component and a second diameter extending from the first length along a second length of the core. The first diameter is larger than the second diameter. The first tube has an inner diameter equal to or less than the first diameter of the core component and greater than the second diameter of the core component. The first tube may have a generally constant outer diameter. The second tube has an inner diameter equal to or less than the second diameter of the core and an outer diameter generally equal to the inner diameter of the first tube. The second tube extends along the second length of the core and the first tube extends along the first length and second length of the core component. The spinal rod may have a generally uniform thickness. The modulus of elasticity of the rod, however, may vary along its length. The spinal rod may have a first modulus of elasticity in the first length and a second modulus of elasticity in the second length. The first modulus of elasticity may then be higher than the second modulus of elasticity.

Additional aspects and features of the present disclosure will be apparent from the detailed description and claims as set forth below.

### BRIEF DESCRIPTION OF THE DRAWINGS

FIG. 1 is a view of a cross section of a spinal rod according to an embodiment of the present invention.

FIG. 2 is a view of a cross section of a spinal rod according to another embodiment of the present invention.

FIG. 3 is an exploded view of parts of a spinal rod according to the embodiment of FIG. 1.

FIG. 4 is the partial side view of a composite spinal rod as shown in the Embodiment of FIG. 1.

FIG. 5 is a partial exploded view of parts of a spinal rod according to the embodiment of FIG. 2.

FIG. 6 is the partial side view of a composite spinal rod as shown in the Embodiment of FIG. 1.

FIG. 7 is a cross section of an embodiment of a spinal rod according to an aspect of the invention.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

It is a feature of an embodiment of the present invention to provide composite rods for use in spinal fixation systems. The composite components may comprise a first core material which may be a metal, metal alloy, a polymer, or a polymeric composite; and a second material formed in a sleeve and selected from the group consisting of resorbable and non-resorbable polymeric materials. In a preferred embodiment, the composite comprises polyetheretherketone tube or sleeve and a metal or metal alloy core.

Polyetheretherketone (PEEK) is a polymer that is commercially available from a number of suppliers and also is available in medical grades that are preferred for use in the embodiments (e.g., PEEK OPTIMA™, commercially available from Invibio Ltd., Lancashire, United Kingdom). The resorbable and non-resorbable polymeric materials, such as PEEK, can be combined with at least one metal or metal alloy in accordance with the embodiments in order to form composite components such as rods and plates for use in spinal fixation systems. Preferred metal and metal alloys for use in the invention include, but are not limited to, titanium, titanium alloys (e.g. Ti-6Al-4V), tantalum, tantalum alloys, stainless steel alloys, cobalt-based alloys, cobalt-chromium alloys, cobalt-chromium-molybdenum alloys, niobium alloys, nickel-titanium alloys (Nitinol), and zirconium alloys.

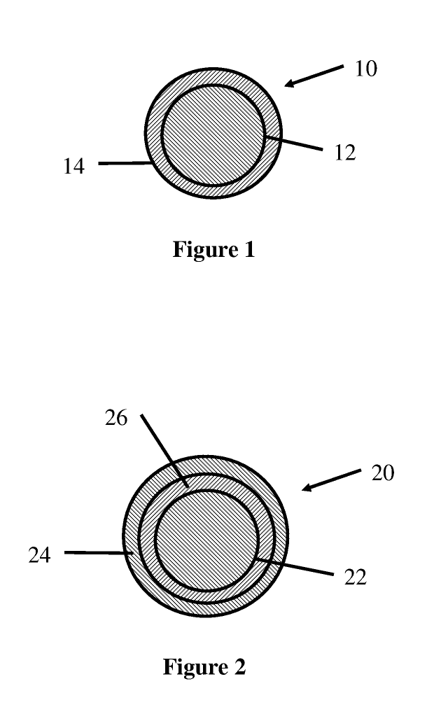

Turning now to FIG. 1, FIG. 1 is a view of a cross section of a spinal rod 10 according to an embodiment of the present invention. The cross section of the spinal rod 10 comprises a central rod or inner core of metal 12 and an outer sleeve or tube of PEEK 14. The diameters of the inner metal core 12 and outer polymer tube 14 may be adjusted to change the modulus of elasticity of the composite. The modulus of elasticity of the construct, though, is bounded by the lower limit of the polymer and the upper limit of the metal. As the diameter of the metal core 12 approaches the total construct diameter, the modulus of elasticity of the construct approaches the modulus of the metal core 12. Similarly, as the thickness of the polymer tube 14 approaches the total construct diameter, the modulus of elasticity of the construct approaches the modulus of the polymer tube 14. This allows, then, a construct having a specific diameter with a modulus of elasticity that may vary based upon the size of the individual components.

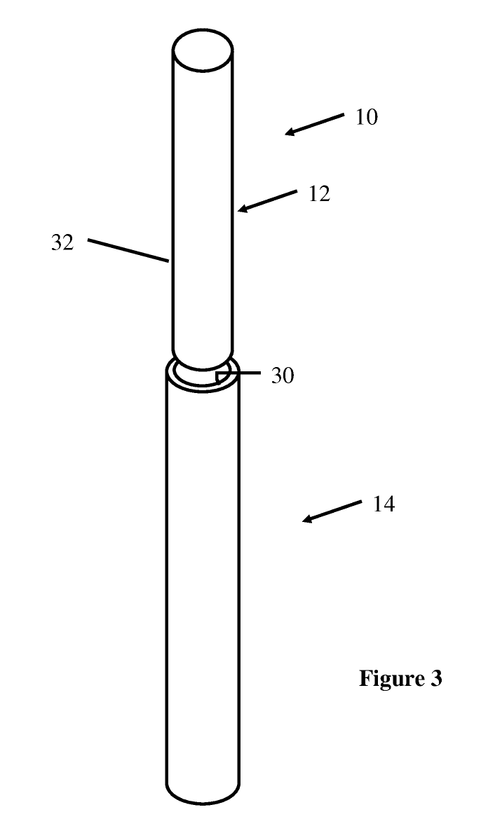

The inner metal core 12 is inserted into the polymer tube 14 by vibrating the core 12 and tube 14 relative to one another. The core 12 may be vibrated, the tube 14 may be vibrated, or both may be vibrated. The vibration allows the core 12 to pass through the inner diameter of the tube 14. As will be described with respect to FIG. 3 below, the interfacial surface between the core 12 and tube 14 may be strengthened by introducing the metal core 12 into the tube 14 in this manner.

Turning now to FIG. 2, FIG. 2 is a view of a cross section of a spinal rod 20 according to another embodiment of the present invention. The cross section of the spinal rod 20 comprises a central rod or inner core of metal 22, a first outer sleeve or tube of PEEK 24 and a second outer sleeve or tube of PEEK 26. The diameters of the inner metal core 22 and outer polymer tubes 24 and 26 may be adjusted to change the modulus of elasticity of the composite. The modulus of elasticity of the construct, though, is bounded by the lower limit of the polymers and the upper limit of the metal. As the diameter of the metal core 22 approaches the total construct diameter, the modulus of elasticity of the construct approaches the modulus of the metal core 22. Similarly, as the thickness of the polymer tube 24 or 26 approaches the total construct diameter, the modulus of elasticity of the construct approaches the modulus of the lower modulus of the polymer tubes 24 or 26.

The polymer tubes 24 and 26 may be of different moduli of elasticity. It may be beneficial to use multiple tubes 24 and 26 as the total thickness of the polymer tubes 24 and 26 increases. As a vibratory force is applied between the core 22 and the polymer tubes 24 and/or 26, the tubes may slightly expand to conform and lock with the inner metal core 22 (for the inner polymer tube 24) or conform and lock with the inner polymer tube 24 (for the inner polymer tube 26). The amount of relative vibration may be reduced by having multiple tubes as the amount of vibration required to introduce the metal core into the tube is a function of the tube thickness, as well as the relative diameters of the core and the tube. Thicker tubes may not be as easily conformable over the core, thus a first tube being advanced and then a second tube advanced over the first tube, may be a more preferable configuration. However, thinner tubes or sleeves may be generally more flexible to bending along the length of the tube, and thus may buckle as the tube is advanced over the inner core. Thus, the tube thickness is preferably thick enough so that the inner core may be moved within the tube without causing ripples in the tube material but thin enough so that the tube is still compliant enough to receive the core.

In addition to the thickness of the tubes, the relative diameters of the tube and the inner core also affect the ease of advancement of the tube over the core. There is a tradeoff between ease of advancement over the inner core and the interfacial conformance force between the core and the tube. The inner diameter of the inner polymer sleeve or tube may be the same diameter or smaller than the outer diameter of the inner metal core. As the difference in diameters between the core and the inner diameter of the sleeve gets larger, the tube is more constrained from advancing over the inner core. However, as the difference becomes less, the amount of the interfacial conformance force generated between the core and the tube is reduced. Higher conformance force makes for stronger pull-out resistance, and the would make the core less likely to separate from the tube.

As is shown in FIG. 3, FIG. 3 is an exploded view of parts of a spinal rod 10 according to the embodiment of FIG. 1. The inner metal core 12 and the outer polymer tube 14 are sized to such that the inner core 12 has a diameter equal or slightly greater than the diameter of the polymer tube 14. An inner wall 30 of the tube 14 has a diameter equal to or smaller than the diameter of an outer wall 32 of the inner core 12. The inner core, then, may be advanced into the tube 14 by a method such as ultrasonic welding.

Ultrasonic welding is a process where high-frequency ultrasonic acoustic vibrations are locally applied to workpieces being held together under pressure to create a solid-state weld. In this application, high frequency ultrasonic acoustic vibrations may be applied to the rod or tubes using the pressure created by the hoop stress between the core and the outer tube in a tight fitting orientation. The polymer may melt at the inner surface during this process, which may further help the conformance between the inner core and the tube.

Preferably, spinal rod composites with a length less than 300 mm (about 12 in) may be formed with this technique. The process is preferably performed at parameters where the frequency is between 20 kHz to 70 kHz, and the amplitude of the vibrations are between 10 μm to 100 μm (0.0004-0.002 in). The cycle time may be a function of the rate of insertion, as well as dependent on the thicknesses and diameters of the rods and tubes. The strength of the weld formed at the junction between the core and the tube is a function of the hoop stresses of the composite formed from the different diameters of the core and tube portions. The weld then forms at the junction between the core and the tube as an interfacial force at the conforming surfaces of the core and tube. This interfacial conformance force, then, maintains the position of the core along the axial length of the tube. As the interfacial conformance force increases, the pull out force of the composite increases.

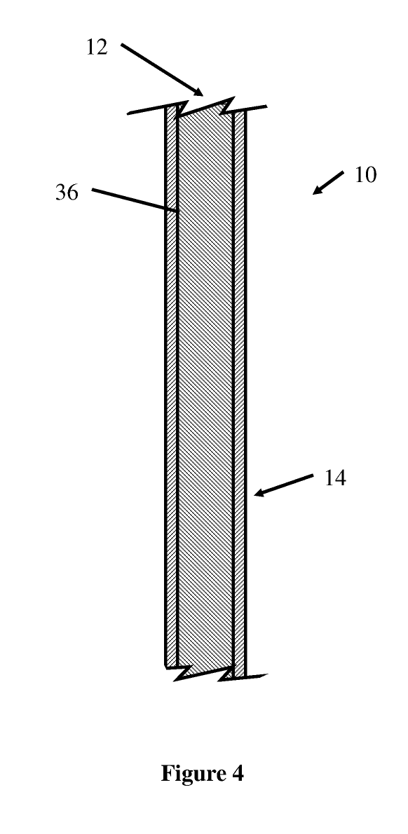

Turning now to FIG. 4, FIG. 4 is the partial side view of a composite spinal rod 10 as shown in the embodiment of FIG. 1. The abutted surface 36 between the inner core 12 and the tube 14 exerts a radially oriented force between the core 12 and the tube 14 to maintain axial position between the core 12 and the tube 14.

The diameters of the inner metal core 12 and outer polymer tube 14 may be adjusted to change the modulus of elasticity of the composite. The modulus of elasticity of the construct, though, is bounded by the lower limit of the polymer and the upper limit of the metal. As the diameter of the metal core 12 approaches the total construct diameter, the modulus of elasticity of the construct approaches the modulus of the metal core 12. Similarly, as the thickness of the polymer tube 14 approaches the total construct diameter, the modulus of elasticity of the construct approaches the modulus of the polymer tube 14. This allows, then, a construct having a specific diameter with a modulus of elasticity that may vary based upon the size of the individual components.

The length of the rod 10, as shown in FIG. 4, is a straight rod. The rod 10, however, may curve along its length. For example, the rod 10 may have a constant radius of curvature along the length. Multiple radii may also be present along the length. These multiple radii may change along the length such that the rod is concave in portions and convex in portions. Such curves may be used to approximate kyphotic and lordotic curves in the spine.

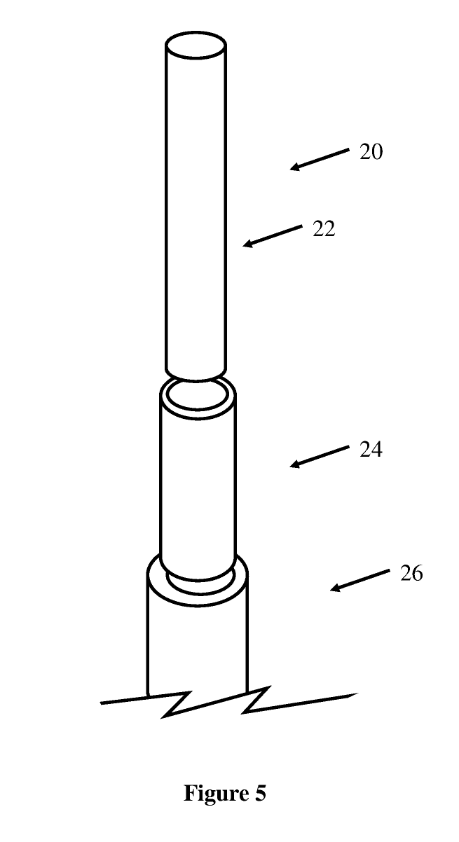

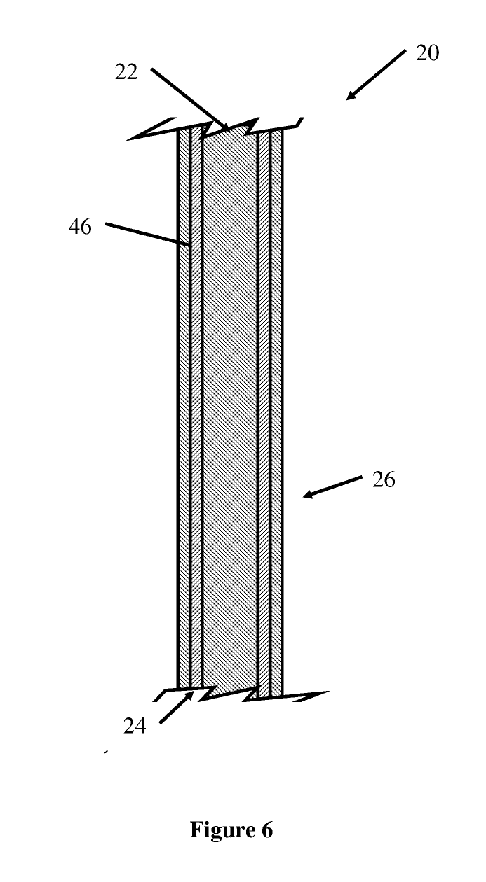

Turning now to FIGS. 5 and 6, FIGS. 5 and 6 correspond to an embodiment similar to the embodiment shown in FIG. 2. FIG. 5 is a partial exploded view of parts of a spinal rod 20 according to the embodiment of FIG. 2. The cross section of the spinal rod 20 comprises a central rod or inner core of metal 22, a first outer sleeve or tube of PEEK 24 and a second outer sleeve or tube of PEEK 26. The diameters of the inner metal core 22 and outer polymer tubes 24 and 26 may be adjusted to change the modulus of elasticity of the composite. The modulus of elasticity of the construct, though, is bounded by the lower limit of the polymers and the upper limit of the metal. As the diameter of the metal core 22 approaches the total construct diameter, the modulus of elasticity of the construct approaches the modulus of the metal core 22. Similarly, as the thickness of the polymer tube 24 or 26 approaches the total construct diameter, the modulus of elasticity of the construct approaches the modulus of the lower modulus of the polymer tubes 24 or 26.

As previously described, the relative diameters of the parts may be sized to allow for ease of advancement of the parts coaxial to one another. The outer tube 26, however, may be sized based on the inner tube 24 diameter either before or after the inner tube has received the core 22. The vibrational energy may be applied to the tubes 24 or 26 or the core 22 serially (thus allowing for a smaller outer tube 26 diameter) or may be applied in parallel thereby requiring the larger inner diameter for the outer sleeve 26. The outer sleeve 26, if advanced in parallel, must conform more than in a composite where the outer tube 26 is not advanced over the inner tube 24 until after the inner tube 24 is advanced to the inner core 22.

While the embodiments have shown one or two tubes in use, in practice, as many tubes as desired for a final thickness may be used. The tubes may have the same modulus of elasticity as other tubes, or may have differing moduli of elasticity depending on the need. As described above, thinner tubes may be easier to advance over the inner metal core as a tradeoff between ease of advancement over the inner core and inner diameter of the inner tube. As that difference in diameter gets larger, the tube is more easily advanced over the inner core. However, as the difference becomes greater, the amount of shrinking required to bond the polymer to the inner core would be greater. Thus, multiple, thinner tubes may be beneficial instead of thicker tubes.

Additionally, the tubes may vary in length and thickness from each other in order to allow for a composite rod having varying thickness along the length of the rod. The thickness of the tubes may be between 0.1 mm and 3 mm, and preferably between 0.25 mm and 1.55 mm. For example, if one end of the rod needs to be thicker, then sleeves having lengths shorter than the length of the core may be used at the end that is desired to be thicker. The additional layers at this end may make the implant thicker at that end, and thus achieve variable thickness along the length of the rod.

Other processes may help to hold the tubes over the core. For example, adhesives may be added between the tube and the core to allow for additional pull out strength between the core and the tube. Other surface features such as surface texturing or surface roughening may also increase the pull out strength between the core and the tube. Such procedures may be physical treatments such as shot-peening or may be chemical processes such as passivation. Other surface features may similarly increase pull out strength such as surface structures like grooves, serrations or spikes that may be cut into or formed on the core surface.

The tubes and rods may also be treated with other agents that may promote healing. Biological and/or pharmacological agents may be added on surfaces or may be embedded in the structures to promote healing by treating inflammation or to promote underlying bone growth or calcification. Antimicrobial agents may also be embedded or added to the surface of the tubes. Agents such as silver may be added to the tube. For example, silver in a concentration by weight of 0.1 to 5% may be added to a PEEK tube in order to help protect against the threat of microbial infection.

One use of rods made according to this invention may be in revision cases. In these types of spinal implant systems, the screws inserted into the vertebra have a rod-capturing portion that is sized according to the original rod diameter. The original rod may need to be a more rigid construct immediately after surgery. Thus, a solid metal (and thus high modulus of elasticity) material may be used. As healing progresses and the vertebra fuse together more completely, the spinal implant system may not need to be as rigid. However, given the other hardware already implanted (namely the rod-capturing portion of the spinal implant system), a similarly sized rod would be the most effective rod to replace within the system. The rod shown above may provide a rod having the same size as the original rod in the system while allowing for a lower modulus of elasticity.

It should be apparent that the composite components provided by the embodiments may take a myriad of different forms or configurations, in accordance with the guidelines provided herein. Therefore, one of skill in the art will appreciate still other configurations for composite spinal fixation components in accordance with the embodiments. For example, the metal and polymer portions of each composite component may have varying thicknesses and geometries, and need not correspond to the relatively uniform thicknesses and geometries depicted in the figures. Additionally, as the different forms change from generally round configurations, the meaning of “diameter” and “diameter” must accordingly adjust from a strict interpretation requiring a circular cross section to allow for the structures of other shapes to fit within these aspects of the invention. Namely, the definitions should submit to an interpretation where an inner core has a centroid and the distance at all polar orientations around that centroid to the inner diameter of the hollow cylindrical tube or sleeve member is greater than the distance to the outer boundary of the inner core before the process to shrink the outer tube has begun. In other words, the shape of the tube should be slidably received over the shape of the core when energized. Accordingly, skilled artisan will appreciate that an infinite number of variations in cross sections of the composite rods provided for by the embodiments may occur, in accordance with the guidelines provided herein.

Although FIGS. 1-7 were illustrated with respect to PEEK/metal composites, according to embodiments of the invention other resorbable and non-resorbable polymeric materials may be used in place of PEEK in the composite structures. For example, a resorbable polymer material such as polylactides (PLA), polyglycolides (PGA), copolymers of (PLA and PGA), polyorthoesters, tyrosine, polycarbonates, and mixtures and combinations thereof may be used in lieu of PEEK. Also, non-resorbable polymeric material such as members of the polyaryletherketone family, polyurethanes, silicone polyurethanes, polyimides, polyetherimides, polysulfones, polyethersulfones, polyamids, polyphenylene sulfides, and mixtures and combinations thereof alternatively may be used in lieu of PEEK. Therefore, a wide variety of composite components may be fabricated in accordance with the embodiments.

PEEK generally has a lower modulus of elasticity and tensile strength than the exemplary metals and metal alloys shown in the table. The differences in physical properties between PEEK and the metals can be advantageously utilized in the embodiments by fabricating the composite spinal fixation rods with appropriate proportions of PEEK and metal, metal alloy, or mixtures thereof to produce a device having the desired physical properties. In this way, composite components can be fabricated having, for example, an average or mean modulus of elasticity different from that of the modulus of elasticity of any of its individual components. For example, consider two rods with the same diameter—the first rod of Ti-6Al-4V and the second rod a composite of Ti-6Al-4V and PEEK. Because a portion of the second rod comprises a material having a lower modulus of elasticity (PEEK), than the modulus of elasticity of Ti-6Al-4V, the second rod will have a lower average or mean modulus of elasticity than the first rod. In general, a composite rod will have average or mean properties, such as average or mean modulus of elasticity, proportionate to the ratio of the components that comprise the rod. One who is skilled in the art will appreciate how to select an appropriate ratio and orientation of the components that make up the systems, rods, plates, and other components based on the desired physical properties, in accordance with the guidelines described herein. For example, other polymeric materials such as those provided herein may be chosen for use in the composite components instead of PEEK, in order to produce composite components having different average or mean properties.

Fabricating composite components of spinal fixation systems may be advantageous because of the ability to produce composite components with average or mean properties not otherwise possible. For example, if a rod of a certain diameter is required for use with a given spinal fixation system, fabricating a composite rod having the required diameter using PEEK and metal composites may yield a composite rod with an average or mean modulus of elasticity not otherwise achievable for the required diameter rod, if fabricated from a non-composite material. Therefore, one advantage provided by the embodiments is that a spinal fixation system component may be fabricated having a different average or mean modulus of elasticity without changing the dimensions or geometry of the component. This may be highly advantageous, for example, where fixation systems are desired to be retrofitted or otherwise customized for use with patients that require a more flexible fixation system, but require components that imitate the dimensions and geometries of the original, non-composite components of the fixation systems. To aid these patients, composite components may be fabricated in accordance with embodiments herein.

In a preferred embodiment, composite spinal fixation rods may be fabricated that have physical properties not otherwise attainable in rods and plates that are composed purely of metals and metal alloys. Preferably, the composite rods and plates have a mean or average modulus of elasticity less than about 75 GPa. Additionally, it is preferable that the composite rods and plates have a mean or average tensile strength less than about 150 MPa. One skilled in the art will be capable of fabricating composite materials comprising PEEK and at least one metal or metal alloy that have one or more of these preferred physical properties.

In another preferred embodiment, composite spinal fixation components may be fabricated comprising PEEK and a metal or metal alloy having a mean or average modulus of elasticity from about 1.2 GPa to about 192 GPa. More preferably, components may be fabricated having a mean or average modulus of elasticity from about 2 GPa to about 100 GPa. Even more preferably, components may be fabricated having a mean or average modulus of elasticity from about 3 GPa to about 50 GPa.

For example, a titanium spinal rod has a modulus of elasticity of about 116 GPa. PEEK has a modulus of elasticity of around 3.6 GPa. For a similarly sized composite rod made of titanium and PEEK, the modulus of elasticity of the composite rod may be reduced by increasing the thickness of the tubes while decreasing the diameter of the metal core. The modulus of elasticity, though, is bounded by the PEEK modulus on the low end and the titanium modulus on the high end. Other material, though, may be used having different moduli, and thus different bounds for the composite modulus of elasticity. For example, a PEEK core may be used with a polyethylene tube to get a much lower average modulus of elasticity.

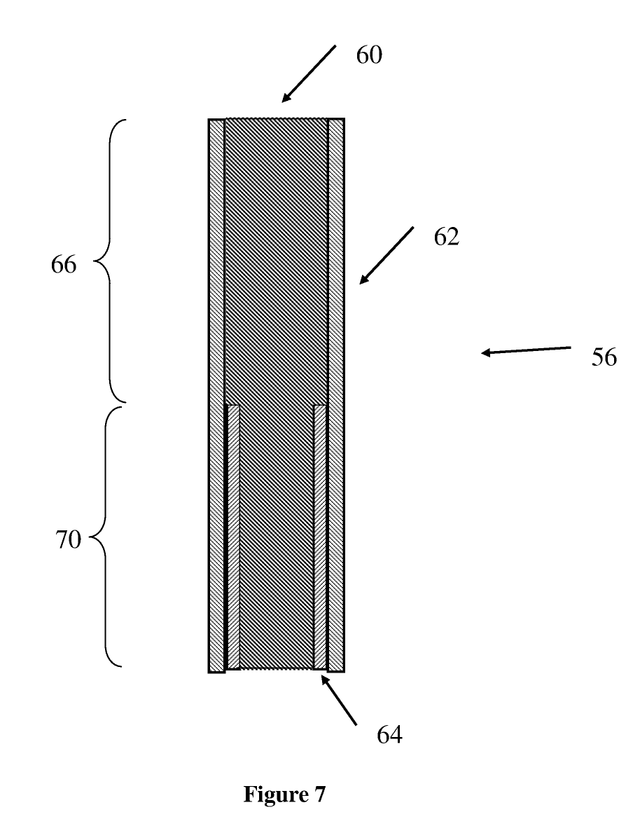

Turning now to FIG. 7, FIG. 7 is a cross section of an embodiment of a spinal rod 56 according to an aspect of the invention. The spinal rod 56 includes a core component 60, a first tube 62, and a second tube 64. The core component 60 has a first diameter extending along a first length 66 of the core component 60 and a second diameter extending from the first length 66 along a second length 70 of the core. The first diameter is larger than the second diameter. The first tube 62 has an inner diameter equal to or less than the first diameter of the core component 60 and greater than the second diameter of the core component 60. The first tube 62 may have a generally constant outer diameter. The second tube 64 has an inner diameter equal to or less than the second diameter of the core and an outer diameter generally equal to the inner diameter of the first tube 62. The second tube 64 extends along the second length 70 of the core and the first tube extends along the first length 66 and second length 70 of the core component 60.

The spinal rod 56 may have a generally uniform thickness. The modulus of elasticity of the rod 56, however, may vary along its length. The spinal rod 56 may have a first modulus of elasticity in the first length 66 and a second modulus of elasticity in the second length 70. The first modulus of elasticity may then be higher than the second modulus of elasticity. Such a construct may be useful when a portion of the spinal rod 56 is used in an area of the spine where the underlying vertebra benefit from a fusion rod, while the second length of the spinal rod 56 is used in a more dynamic area of the spine, where the surgeon may wish to preserve some motion. Because the rod is generally uniform in cross section between the first and second lengths 66 and 70, the same receivers may be used with both portions of the rod. This may limit required inventory, decrease surgical time (as surgeons would not be required to size and position varying receivers) and improve performance by providing both fusion capability and motion preserving capability in the same rod.

Previous composite spinal fixation rods have been formed by utilizing a metal injection molding (MIM) technique to fabricate the metallic portion, and an injection molding technique to fabricate the non-metallic, or polymeric portion. Disadvantages of the MIM process include requiring application of several hundred tons of pressure to a mold. This results in high tooling costs and precision processes.

In another embodiment, the second material may be mixed or combined with a first material comprising a metal or metal alloy. Thus, each component may be a composite comprising the first material and the second material which may be used to fabricate various composite rods as has been described herein in regards to PEEK. The composites comprising a first material and second material as described herein may be advantageously used to fabricate spinal fixation system components having average or mean properties not otherwise attainable for a given dimension or size when using non-composite materials to fabricate the components.

The foregoing detailed description is provided to describe the invention in detail, and is not intended to limit the invention. Those skilled in the art will appreciate that various modifications may be made to the invention without departing significantly from the spirit and scope thereof.

Furthermore, it is understood that any spatial references, such as “first,” “second,” “exterior,” “interior,” “superior,” “inferior,” “anterior,” “posterior,” “central,” “annular,” “outer,” and “inner,” are for illustrative purposes only and can be varied within the scope of the disclosure.

1. A spinal rod composite comprising:
a core component having a diameter and an axial length; and
a tube having a diameter equal to or less than the diameter of the core component, wherein a vibrational energy is applied between the core and the tube such that the core is received within the tube and the tube is advanced along the axial length of the tube, the spinal rod composite then having facial conformance forces maintaining the tube position along the axial length of the core.

2. The spinal rod of claim 1, wherein the tube includes a plurality of nested tubes.

3. The spinal rod of claim 2, wherein a tube of the plurality of nested tubes has a different modulus of elasticity than another tube of the plurality of nested tubes.

4. The spinal rod of claim 1, wherein the tube is formed from a PEEK material.

5. The spinal rod of claim 1, wherein the core component is a metal formed from titanium, a titanium alloy, cobalt chrome, or a stainless steel alloy.

6. The spinal rod of claim 5, wherein the tube is a polymeric material having a different modulus of elasticity than the metal core component.

7. The spinal rod of claim 1, wherein the tube has a thickness between 0.1 mm and 3 mm.

8. The spinal rod of claim 7, wherein the tube has a thickness between 0.25 mm and 1.5 mm.

9. The spinal rod of claim 1, wherein the outer surface of the core further comprises surface features.

10. The spinal rod of claim 1, wherein the core is curved along the length.

11. The spinal rod of claim 1, wherein the tube further comprises an antimicrobial agent.

12. The spinal rod of claim 11, wherein the tube is made of a PEEK material and the antimicrobial agent is silver embedded in the PEEK tube in a concentration by weight of between 0.1 and 5%.

13. A method of forming a composite spinal rod, comprising the steps of:
advancing a core into a tube, wherein the tube has an inner diameter less than the diameter of the core; and
vibrating the core relative to the tube as the advancing step occurs.

14. The method of claim 14, further comprising the step of advancing the core and tube composite into a second tube.

15. The method of claim 14, wherein the vibrating step includes vibrating the core relative to the tube at a frequency between 20 kHz and 70 kHz.

16. The method of claim 14, wherein the vibrating step includes vibrating the core relative to the tube at an amplitude between 10 μm to 100 μm.

17. The method of claim 11, further comprising the step of shot-peening the surface of the core.

18. A spinal rod comprising:
a core component having a first diameter extending along a first length of the core component and a second diameter extending from the first length along a second length of the core component, the first diameter being greater than the second diameter;
a first tube having an inner diameter equal to or less than the first diameter of the core component and greater than the second diameter of the core component, the first tube further having a generally constant outer diameter; and
a second tube having an inner diameter equal to or less than the second diameter of the core component and an outer diameter generally equal to the inner diameter of the first tube,
wherein the second tube extends along the second length of the core component and the first tube extends along the first and second lengths of the tube such that the spinal rod has a generally uniform thickness, the spinal rod having a first modulus of elasticity in the first length and a second modulus of elasticity in the second length, the first modulus of elasticity being higher than the second modulus of elasticity.

19. The spinal rod of claim 18, further comprising an antimicrobial agent, wherein the first and second tubes are made of PEEK material, the antimicrobial agent is silver embedded in the PEEK tube in a concentration by weight of between 0.1 and 5%.

20. The spinal rod of claim 18 wherein the core is curved along the first length.
